(19) United States
(12) Patent Application Publication
(10) Pub. No.: US 2026/0004552 A1
(43) Pub. Date: Jan. 1, 2026

(54) NON-VISIBLE-SPECTRUM LIGHT IMAGE-BASED TRAINING AND USE OF A MACHINE LEARNING MODEL

(71) Applicant: Intuitive Surgical Operations, Inc., Sunnyvale, CA (US)

(72) Inventors: Anthony M. Jarc, Johns Creek, GA (US); Theodore W. Rogers, Alameda, CA (US)

(21) Appl. No.: 18/867,593

(22) PCT Filed: Jun. 14, 2023
(86) PCT No.: PCT/US2023/025290

§ 371 Date: Nov. 20, 2024

Related U.S. Application Data
(60) Provisional application No. 63/352,813, filed on Jun. 16, 2022.

(51) Int. Cl. G06V 10/60; A61B 34/10; G06N 20/00; G06T 7/00; G06V 10/44; G16H 30/20; G16H 30/40

(52) U.S. Cl. CPC G06V 10/60; A61B 34/10; G06N 20/00; G06T 7/0012; G06V 10/44; G16H 30/20; G16H 30/40; G06T 2207/10064

(57) ABSTRACT

An illustrative system may access a first image sequence captured by an imaging device during a medical procedure, the first image sequence comprising first images. the first images based on illumination of a scene associated with the medical procedure using visible-spectrum light: access a second image sequence captured by the imaging device during the medical procedure. the second image sequence comprising second images. the second images based on illumination of the scene using non-visible spectrum light: and provide the first image sequence and the second image sequence to a machine learning module.

### RELATED APPLICATIONS

The present application claims priority to U.S. Provisional Patent Application No. 63/352,813, filed Jun. 16, 2022, the contents of which is hereby incorporated by reference in its entirety.

### BACKGROUND INFORMATION

Light-based image data captured during medical procedures has many uses, during such procedures and after. For example, medical image data from an endoscope can be displayed during a medical procedure to help medical personnel carry out the procedure. As another example, medical image data captured during a medical procedure can be used as a control signal for computer-assisted medical systems. As another example, medical image data captured during a medical procedure may also be used after the medical procedure for post-procedure evaluation, diagnosis, instruction, and so forth.

A variety of illuminating and image-sensing technologies have been used to capture images of medical procedures. Visible-spectrum illuminants and image sensors have been used to capture color (white light) images of medical procedures. Non-visible-spectrum image sensors, sometimes paired with non-visible-spectrum illuminants, have been used to capture non-visible-spectrum images of medical procedures.

### SUMMARY

The following description presents a simplified summary of one or more aspects of the systems and methods described herein. This summary is not an extensive overview of all contemplated aspects and is intended to neither identify key or critical elements of all aspects nor delineate the scope of any or all aspects. Its sole purpose is to present one or more aspects of the systems and methods described herein as a prelude to the detailed description that is presented below.

An illustrative system includes a memory storing instructions; and one or more processors communicatively coupled to the memory and configured to execute the instructions to perform a process comprising: accessing a first image sequence captured by an imaging device during a medical procedure, the first image sequence comprising first images, the first images based on illumination of a scene associated with the medical procedure using visible-spectrum light; accessing a second image sequence captured by the imaging device during the medical procedure, the second image sequence comprising second images, the second images based on illumination of the scene using non-visible spectrum light; and processing the first image sequence and the second image sequence using a machine learning module.

Another illustrative system includes a memory storing instructions; and one or more processors communicatively coupled to the memory and configured to execute the instructions to perform a process comprising: accessing a first image sequence captured by an imaging device during a medical procedure, the first image sequence comprising first images, the first images based on illumination of a scene associated with the medical procedure using visible-spectrum light; providing the first images to a trained machine learning model, wherein the trained machine learning model has been trained using a second image sequence comprising second images; and performing, based on an output of the machine learning model, an operation with respect to the first image sequence.

Another illustrative system includes a memory storing instructions; and one or more processors communicatively coupled to the memory and configured to execute the instructions to perform a process comprising: accessing a first image sequence captured by an imaging device during a medical procedure, the first image sequence comprising first images, the first images based on illumination of a scene associated with the medical procedure by visible-spectrum light; providing the first images to a trained machine learning model, wherein the trained machine learning model has been trained using a second image sequence comprising second images; and generating, based on an output of the machine learning model, a prediction.

An illustrative method includes: accessing a first image sequence captured by an imaging device during a medical procedure, the first image sequence comprising first images, the first images based on illumination of a scene associated with the medical procedure using visible-spectrum light; accessing a second image sequence captured by the imaging device during the medical procedure, the second image sequence comprising second images, the second images based on illumination of the scene using non-visible spectrum light; and processing the first image sequence and the second image sequence using a machine learning module.

Another illustrative method includes: accessing a first image sequence captured by an imaging device during a medical procedure, the first image sequence comprising first images, the first images based on illumination of a scene associated with the medical procedure using visible-spectrum light; and providing the first images to a trained machine learning model, wherein the trained machine learning model has been trained using a second image sequence comprising second images; and performing, based on an output of the machine learning model, an operation with respect to the first image sequence.

Another illustrative method includes: accessing a first image sequence captured by an imaging device during a medical procedure, the first image sequence comprising first images, the first images based on illumination of a scene associated with the medical procedure using visible-spectrum light; providing the first images to a trained machine learning model, wherein the trained machine learning model has been trained using a second image sequence comprising second images; and generating, based on an output of the machine learning model, a prediction.

An illustrative non-transitory computer-readable medium may store instructions that, when executed, direct a processor of a computing device to: access a first image sequence captured by an imaging device during a medical procedure, the first image sequence comprising first images, the first images based on illumination of a scene associated with the medical procedure using visible-spectrum light; access a second image sequence captured by the imaging device during the medical procedure, the second image sequence comprising second images, the second images based on illumination of the scene using non-visible spectrum light; and process the first image sequence and the second image sequence using a machine learning module.

Another illustrative non-transitory computer-readable medium may store instructions that, when executed, direct a processor of a computing device to: access a first image sequence captured by an imaging device during a medical procedure, the first image sequence comprising first images, the first images based on illumination of a scene associated with the medical procedure using visible-spectrum light; and provide the first images to a trained machine learning model, wherein the trained machine learning model has been trained using a second image sequence comprising second images; and perform, based on an output of the machine learning model, an operation with respect to the first image sequence.

Another illustrative non-transitory computer-readable medium may store instructions that, when executed, direct a processor of a computing device to: access a first image sequence captured by an imaging device during a medical procedure, the first image sequence comprising first images, the first images based on illumination of a scene associated with the medical procedure using visible-spectrum light; provide the first images to a trained machine learning model, wherein the trained machine learning model has been trained using a second image sequence comprising second images; and generate, based on an output of the machine learning model, a prediction.

### BRIEF DESCRIPTION OF THE DRAWINGS

The accompanying drawings illustrate various embodiments and are a part of the specification. The illustrated embodiments are merely examples and do not limit the scope of the disclosure. Throughout the drawings, identical or similar reference numbers designate identical or similar elements.

FIG. 1 shows a system for capturing images of a scene associated with a medical procedure.

FIG. 2 shows a machine learning module.

FIG. 3 shows a machine learning data flow.

FIG. 4 shows an embodiment in which labels are generated for a first image sequence based on a second image sequence.

FIG. 5 shows an embodiment using image blending.

FIG. 6 shows examples of outputs of a trained machine learning model.

FIG. 7 shows a computer-assisted medical system.

FIG. 8 shows an illustrative computing device.

### DETAILED DESCRIPTION

Techniques for using non-visible-spectrum images to enable machine learning about visible-spectrum images are described herein. Given a first sequence of images (e.g., visible-spectrum images) of a scene associated with a medical procedure and a second sequence of images (e.g., non-visible-spectrum images) of the scene, the first sequence and the second sequence can both be used, directly or indirectly, to train a machine learning model that can produce outputs not possible when only one or the other is used for training. As described herein, the outputs of the machine learning module may be used to perform various operations with respect to the first image sequence and/or a computer-assisted medical system, which may advantageously provide various benefits as described herein.

As used herein, “visible-spectrum image” and “visible-spectrum video” refer to images and video whose pixel values represent sensed intensities of visible-spectrum light. “Non-visible-spectrum image” and “non-visible-spectrum video” refer to images and video whose pixel values represent sensed intensities of non-visible-spectrum light. For brevity, “image” will be used herein to refer to both images and video. Illustrative non-visible-spectrum images include fluorescence images, hyperspectral images, and other types of images that do not rely solely on visible-spectrum illumination. For example, fluorescence images are images of light fluoresced from matter when the matter is illuminated by a non-visible-spectrum illuminant. Infrared images are another type of non-visible-spectrum image. Infrared images are images captured by sensors that can sense light in an infrared wave range. For example, the infrared light may include light emitted by illuminated fluorophores.

As used herein, a “label” refers to any type of data indicative of an object or other feature represented in an image including, but not limited to, graphical or text-based annotations, tags, highlights, augmentations, and overlays. A label applied to an image may be embedded as metadata in an image file or may be stored in a separate data structure that is linked to the image file. A label can be presented to a user, for example, as an augmentation to the image, or may be utilized for other purposes that do not necessarily involve presentation such as training of a machine learning model.

As used herein, a “medical procedure” can refer to any procedure in which manual and/or instrumental techniques are used on a patient to investigate, diagnose, or treat a physical condition of the patient. Additionally, a medical procedure may refer to any non-clinical procedure, e.g., a procedure that is not performed on a live patient, such as a calibration or testing procedure, a training procedure, and an experimental or research procedure.

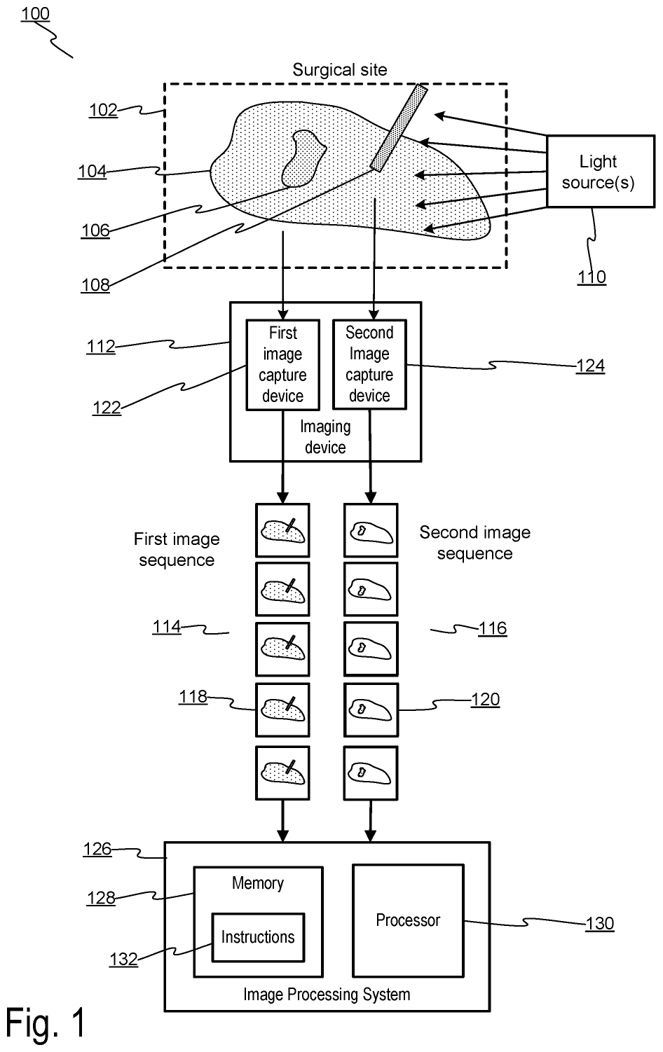

FIG. 1 shows a system 100 for capturing images of a scene 102 associated with a medical procedure. Scene 102 may include a surgical area associated with a body on or within which the medical procedure is being performed (e.g., a body of a live animal, a human or animal cadaver, a portion of human or animal anatomy, tissue removed from human or animal anatomies, non-tissue work pieces, physical training models, etc.). For example, the scene 102 may include various types of tissue (e.g., tissue 104), organs (e.g., organ 106), and/or non-tissue objects (e.g., object 108) such as instruments, objects held or manipulated by instruments, etc.

One or more light sources 110 may illuminate the scene 102. As noted above, the light sources 110 might include any combination of a white light source, a narrow-band light source (whether in the visible spectrum or not, e.g., an ultraviolet lamp), a laser, an infrared light emitting diode (LED), etc. If fluoresced light is to be captured, the type of light source may depend on the fluorescing agent or protein being used during the medical procedure. In some implementations, a light source might provide light in the visible spectrum but the fluoresced light that it induces may be out of the visible spectrum.

Further regarding fluoresced light, in some implementations of the system 100, a light source for fluorescence illumination (i.e., an excitation light source) may have any wavelength outside the visible spectrum. For example, a fluorescence illuminant, such as indocyanine green (ICG), may produce light with a wavelength in an infrared radiation region (e.g., about 700 nm to 1 mm), such as a near-infrared (“NIR”) radiation region (e.g., about 700 nm to 950 nm), a short-wavelength infrared (“SWIR”) radiation region (e.g., about 1,400 nm to 3,000 nm), or a long-wavelength infrared (“LWIR”) radiation region (e.g., about 8,000 nm to 15,000 nm). Additionally, or alternatively, the fluorescence illuminant may output light with a wavelength of about 350 nm or less (e.g., ultraviolet radiation). In some implementations, the fluorescence illuminant may be specifically configured for optical coherence tomography imaging.

The system 100 also includes an imaging device 112. The imaging device 112 receives light reflected, emitted, and/or fluoresced from the subject of the medical procedure and converts the received light to image data. The imaging device 112 senses light from the scene 102 and outputs a first image sequence 114 and a second image sequence 116 of the scene. The first image sequence 114 may be a sequence of visible-spectrum images 118 of light sensed in the visible spectrum. The second image sequence 116 may be a sequence of non-visible-spectrum images 120 of light sensed in a non-visible-spectrum. For example, the second image sequence may be based on illumination of the scene and/or a scene associated with a different medical procedure using non-visible spectrum light. Alternatively, the second image sequence may include visible light images having labels generated based on non-visible light images.

The image sequences shown in FIG. 1 may be in the form of individual images, an encoded video stream, etc. As shown in FIG. 1, because the image sequences are from different spectrums (or partially non-overlapping spectrums), the content of the respective image sequences may differ; some features of the site may be represented in one sequence and not the other.

The imaging device 112 may have a first image capture device 122 and a second image capture device 124. Either image capture device may be any type of device capable of converting photons to an electrical signal, for example a charge-coupled device (CCD), a complementary metal oxide semiconductor (CMOS) sensor, a photo multiplier, etc. Regardless of the type of image capture devices used, the imaging device 112 may be configured to sense light in both the visible spectrum and outside the visible spectrum, as noted above. In some embodiments, the first image capture device 122 senses light in the visible spectrum, and the second image capture device 124 senses light in a non-visible spectrum. The first image capture device 122 and the second image capture device 124 may be separate sensors within a single camera, or they may be separate sensors in separate respective cameras. In some embodiments, the imaging device 112 may include only one image capture device (e.g., one sensor), and the image capture device is capable of concurrently sensing in the visible spectrum and in one or more non-visible spectrums. For example, some image sensors are capable of simultaneously sensing in the visible spectrum and in an infrared spectrum. In other embodiments, the imaging device 112 may be a stereoscopic camera and may have two cameras each capable of sensing in the visible spectrum and a non-visible spectrum. In some embodiments, the imaging device 112 and the light sources 110 may be part of (or optically connected with) an endoscope.

In one embodiment, the first image capture device 122 may continuously capture the first image sequence 114 as video data of the medical procedure, and the second image capture device 124 may capture the images of the second image sequence 116 intermittently. For example, the first image capture device 122 might capture a video frame (first image 118) every 60th of a second and the second image capture device 124 might capture a second image 120 once every second. This is described more fully in co-pending U.S. Provisional Patent Application No. ______, entitled “Non-visible-spectrum Light Image-based Operations for Visible-spectrum Images” and filed the same day as the present application and incorporated herein by reference in its entirety.

As shown in FIG. 1, the image processing system 126 may be configured to access (e.g., receive) the first image sequence 114 and the second image sequence 116 to perform various operations with respect to the image sequences, as described below.

The image processing system 126 may be implemented by one or more computing devices and/or computer resources (e.g., processors, memory devices, storage devices, etc.) as may serve a particular implementation. As shown, the image processing system 126 may include, without limitation, a memory 128 and a processor 130 selectively and communicatively coupled to one another. The memory 128 and the processor 130 may each include or be implemented by computer hardware that is configured to store and/or process computer software. Various other components of computer hardware and/or software not explicitly shown in FIG. 1 may also be included within the image processing system 126. In some examples, the memory 128 and the processor 130 may be distributed between multiple devices and/or multiple locations as may serve a particular implementation.

The memory 128 may store and/or otherwise maintain executable data used by the processor 130 to perform any of the functionality described herein. For example, the memory 128 may store instructions 132 that may be executed by the processor 130. The memory 128 may be implemented by one or more memory or storage devices, including any memory or storage devices described herein, that are configured to store data in a transitory or non-transitory manner. The instructions 132 may be executed by the processor 130 to cause the image processing system 126 to perform any of the functionality described herein. The instructions 132 may be implemented by any suitable application, software, code, and/or other executable data instance. Additionally, the memory 128 may also maintain any other data accessed, managed, used, and/or transmitted by the processor 130 in a particular implementation.

The processor 130 may be implemented by one or more computer processing devices, including general purpose processors (e.g., central processing units (CPUs), graphics processing units (GPUs), microprocessors, etc.), special purpose processors (e.g., application specific integrated circuits (ASICs), field-programmable gate arrays (FPGAs), etc.), digital signal processors, or the like. Using the processor 130 (e.g., when the processor 130 is directed to perform operations represented by the instructions 132 stored in the memory 128), the image processing system 126 may perform various operations as described herein.

Various implementations of the image processing system 126 will now be described with reference to the figures and how the image processing system 126 may be configured to implement machine learning techniques. The various modules described herein may be included in the image processing system 126 and may be implemented by any suitable combination of hardware and/or software. As such, the modules represent various functions that may be performed by the image processing system 126 alone or in combination with any of the other functions described herein as being performed by the image processing system 126 and/or a component thereof.

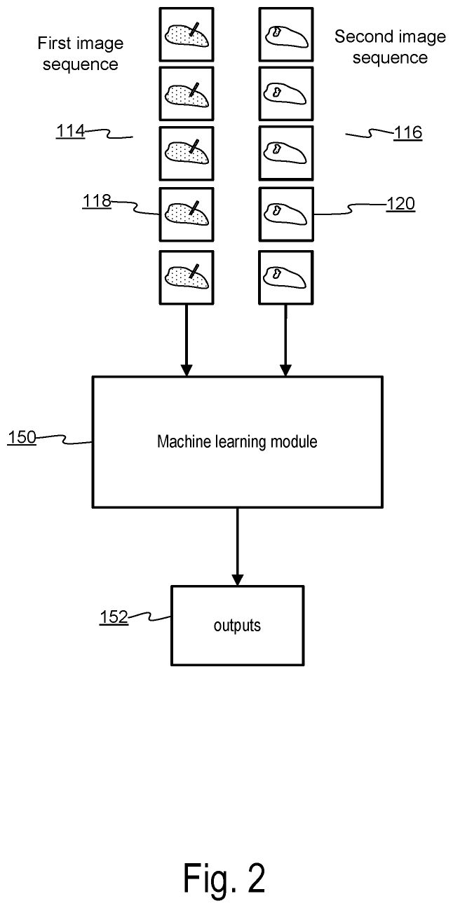

FIG. 2 shows a machine learning module 150 that may be implemented by image processing system 126. The machine learning module 150 receives the first image sequence 114, the second image sequence 116, or both. In some embodiments, the machine learning module 150 is a supervised machine learning algorithm for producing and training machine learning models based on either or both of the image sequences. In some embodiments, more than two image sequences may be input into the machine learning module 150, in which case the machine learning module 150 may be trained using one or more of the image sequences.

In some embodiments, the machine learning module 150 may be implemented by one or more of a regression algorithm, a decision-tree algorithm, a random forest algorithm, a logistic regression algorithm, a support vector machine algorithm, a naïve Bayes classifier algorithm, a linear regression algorithm, a neural network algorithm, and so forth. In embodiments, where the machine learning module 150 is a machine learning algorithm, the outputs 152 of the machine learning module 150 are trained machine learning models.

In other embodiments, the machine learning module 150 is a machine learning model that has been trained based on one or more image sequences. In these embodiments, the outputs 152 of the machine learning module are predictions based on working data, which may be data like the first image sequence (e.g., visible-spectrum images), like the second image sequence (e.g., non-visible-spectrum images), or both. As discussed below, the predictions (outputs 152) of the machine learning module 150 (model) may be predicted images (e.g., synthetic images), features predicted in the images of the working data (e.g., types of tissues or objects), predicted categories of the working data images (e.g., predicted stages of a medical procedure), predicted segmentations, and others that are discussed below.

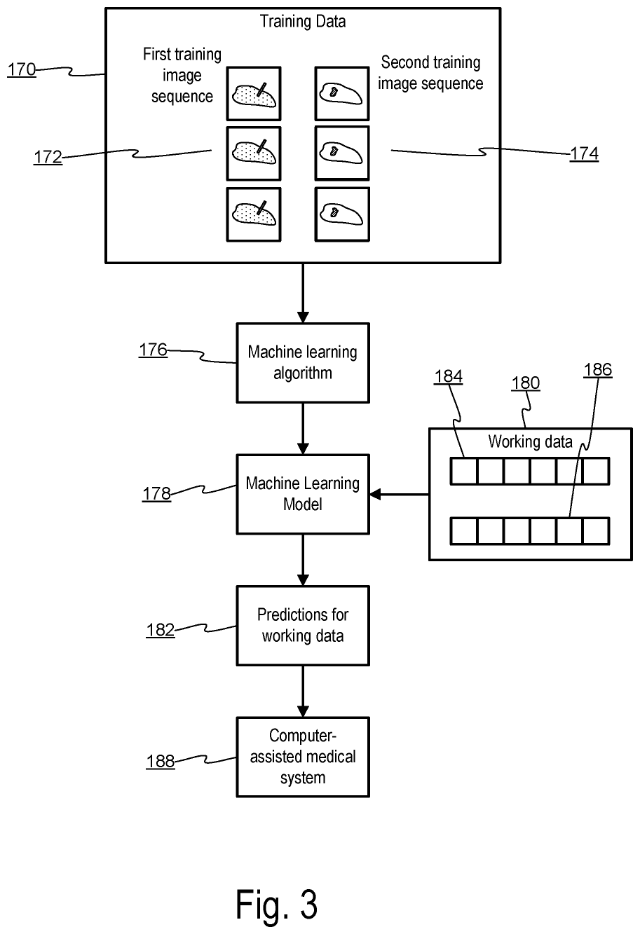

FIG. 3 shows a machine learning data flow. Training data 170 may include a first training image sequence 172 and a second training image sequence 174. Although the training data 170 includes two image sequences of different types of images (e.g., visible-spectrum images and non-visible-spectrum images, respectively), in varying embodiments, the training data 170 that is passed to a machine learning algorithm 176 may include one of the image sequences (e.g., modified or labeled according to the other image sequence), both of the image sequences (e.g., modified or labeled one according to the other), a third image sequence (not shown) of images derived from both image sequences (e.g., a sequence of hybrid images) or that includes a sequence of non-visible light images based on imaging in a different wavelength than the second image sequence, an image sequence that includes hyperspectral images across a wide range of wavelengths, etc. Variations of the training data 170 are discussed further below.

The machine learning algorithm 176 receives the training data 170 and produces a machine learning model 178. The type of model produced by the machine learning algorithm 176 will depend on which machine learning algorithm is used in any given implementation.

The trained machine learning model 178 is used by inputting working data 180 to the machine learning model 178, which in turn generates and outputs predictions 182. Like the training data 170, the working data 180 may include a first working image sequence 184 and a second working image sequence 186. Although the working data 180 may include two image sequences of different types of images (e.g., visible-spectrum images and non-visible-spectrum images, respectively), as with the training data 170, in varying embodiments, the working data 180 that is passed to the machine learning model 178 may be one of the working data image sequences (e.g., modified or labeled according to the other image sequence), both of the working image sequences (e.g., possibly modified or labeled one according to the other), a third working image sequence (not shown) of images derived from both working image sequences (e.g., a sequence of hybrid images), etc. In any case, the machine learning model 178 produces the predictions 182 based on the working data 180.

In some examples, the number of sequences in working data 180 may be the same as the number of sequences in training data 170. In some alternative examples, the number of sequences in working data 180 may be different than the number of sequences in training data 170. For example, the training data 170 may include visible light images and non-visible light images that are used to label the visible light images. The machine learning model 178 may thus be trained using labeled visible light images to produce predictions based on working data 180 that, for example, only includes visible light images.

The predictions 182 may be labels indicative of features (e.g. tissue types, anatomical features, detected or recognized objects, etc.) in images of the working data 180, segmentations of images in the working data 180, images synthesized from images in the working data 180 (synthetic images), features extracted from images in the working data 180, predicted categories of images (or features thereof) in the working data 180, geometry of the scene represented by the images in the working data 180, and others that are discussed later.

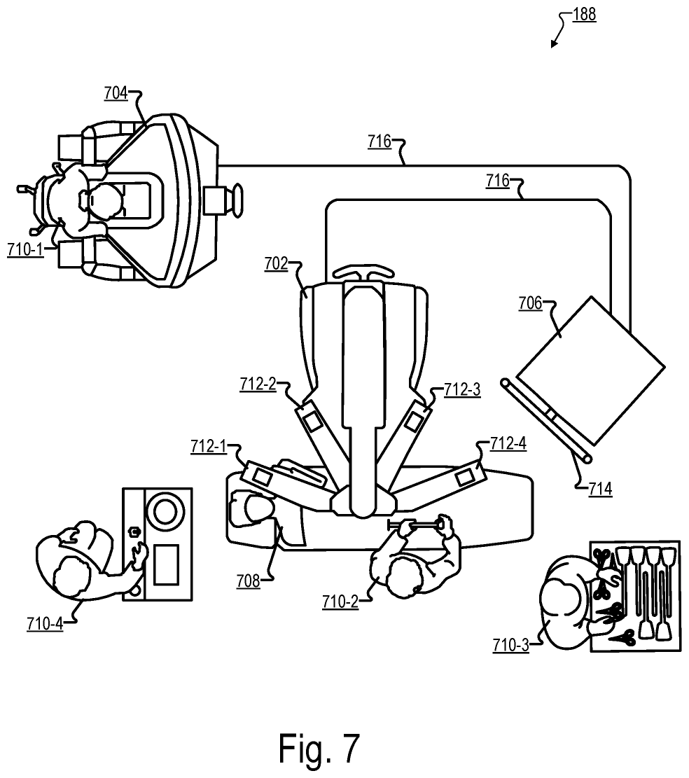

In some embodiments, the predictions 182 stand on their own as a useful product without further computation thereupon. For example, the predictions 182 may be used for post-procedure evaluation (e.g., predictions of stages of a medical procedure), human instruction, medical diagnosis, etc. In some embodiments, the predictions 182 about the working data 180 are provided to a computer-assisted medical system 188 (discussed below with reference to FIG. 7) which may use the predictions 182 in various ways. For example, the predictions 182 may be images displayed by the computer-assisted medical system 188. The predictions 182 may be used to control movement of various components of or connected to the computer-assisted medical system (e.g., by controlling a manipulator arm of the computer-assisted medical system, preventing the movement of instrumentation near predicted anatomical features, etc.). The predictions 182 may be used to inform the content of a user interface of the computer-assisted medical system 188, e.g., when to display indicia and/or graphics of sub-surface (or intra-tissue) anatomy or labels of anatomical features. The predictions 182 may be used to control an imaging mode of the computer-assisted medical system 188, trigger video capture, and so forth.

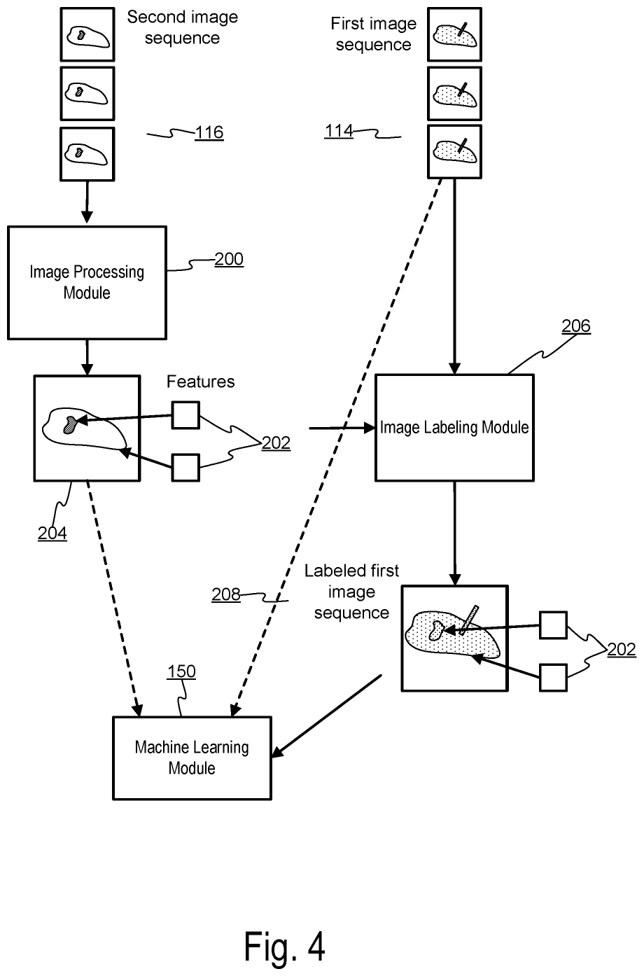

FIG. 4 shows an embodiment in which labels are generated for a first image sequence 114 based on a second image sequence 116 (e.g., based on features in the second image sequence 116). The first image sequence 114 and second image sequence 116 may be visible-spectrum images and non-visible-spectrum images, respectively, as discussed above.

In the embodiment shown in FIG. 4, the second image sequence 116 is passed to an image processing module 200. The image processing module 200 may be coded with one or more image processing algorithms to perform image analysis on the images in the second image sequence 116. The image processing module 200 may perform image processing operations such as feature detection and identification, feature enhancement, etc. Features 202 may be detected and identified based on known traits of pixels for the particular type of non-visible-spectrum imaging technology (e.g., fluorescence imaging) used for the second image sequence 116. For example, pixels having color or intensity values within one color range or intensity range may correspond to one type of organ (or tissue, or object), and pixels having color or intensity values withing another color range or intensity range may correspond to another type of organ, tissue, object, etc. Individual regions (patches) of contiguous like-type pixels may be respectively labeled, individual pixels may be labeled according to their types, boxes (or other shapes) containing a threshold ratio of like-type pixels may be labeled, etc. In some embodiments, labels may be associated with the individual second images themselves. For example, second images determined to contain one or more types of tissues, organs, or objects may be labeled accordingly. A second image having pixel values indicating the presence of cancerous tissue may be labeled accordingly. Because the image processing module 200 receives a sequence of images, the image analysis performed by image processing module 200 may, in some examples, include inter-image analysis.

When a second image is finished being processed by the image processing module 200, the image processing module 200 outputs a sequence of labeled second images 204, which, in one embodiment, is provided to an image labeling module 206. The image labeling module 206 may also receive the first image sequence 116. The image labeling module 206 may assure that a given labeled second image 204 is correlated with an image in the first image sequence 116 (the labeled second image 204 may correlate with, and provide labels for, one or more first images, but for brevity only one first image will be mentioned). This may involve steps such as comparing image timestamps to match a labeled second image 204 with the first image. In some embodiments, when a labeled second image 204 has been paired with the first image, geometric transforms (e.g., affine, scaling) may be performed on either or both images to geometrically align the labeled second image 204 with its corresponding first image (i.e., any two corresponding pixels in respective first and second images represent a same point of the scene). Note that time-pairing and transform operations may be omitted in some embodiments; pairing may be implicit (i.e., the flow of images to the image labeling module 206 may implicitly match time-correlated images) and geometric misalignment may not be present or may not affect labeling of the first image.

Regardless of whether any time-pairing or transform operations are performed, the image labeling module 206 labels the first image according to the labels of the labeled second image 204. In cases where the labeled second image 204 is geometrically aligned with the first image (i.e., the first and second images represent the same scene on a pixel-by-pixel basis), then the feature-labels of the labeled second image 204 may translate to the first image directly. In some embodiments, the image labeling module 206 may perform object/feature detection, segmentation, etc., and then attempt to match features in the first image with features in the labeled second image 204, for example based on the shape, intensities, location, etc. of features. When a feature in the labeled second image 204 matches a feature in the first image then the feature in the first image is labeled according to the matching feature in the labeled second image 204. As noted above, the labels of the second image may be associated with the image but not any particular features thereof, in which case the first image itself is labeled accordingly.

Over time, the labeling process discussed above is repeated for subsequent first and second images, thus forming a labeled first image sequence 208. The images in the first image sequence 116 are then provided to the machine learning module 150, i.e., the labeled first image sequence 208 is provided to a machine learning algorithm to train a machine learning model or is provided to a trained machine learning model which computes predictions for the respective labeled first image sequence 208.

In some embodiments, the image labeling module 206 is omitted, as well as the labeling of the first images. Instead, the second image sequence is labeled by the image processing module 200 as discussed above. And, as indicated by the dashed arrows in FIG. 4, the labeled second images 204 and the first image sequence 116 are passed to the machine learning module 150. Assuming that the machine learning module 150 processes pairs of first and second images at the same time, the first and second images provide a combined signal of correlated visible-spectrum image data and labeled non-visible-spectrum image data for either machine learning training or prediction, as the case may be. If the machine learning module 150 is a model trained using labeled second images and first images then it may output predictions about the first images. Such predictions might be predicted labels of features in the first and/or second images, enhanced first images, segmentations of first images, categories of first images, etc.

In some examples, image processing module 200 and image labeling module 206 may be implemented by (e.g., as sub-modules) of machine learning module 150. Hence, in some examples, machine learning module 150 may be configured to perform the operations described herein as being performed by image processing module 200 and image labeling module 206.

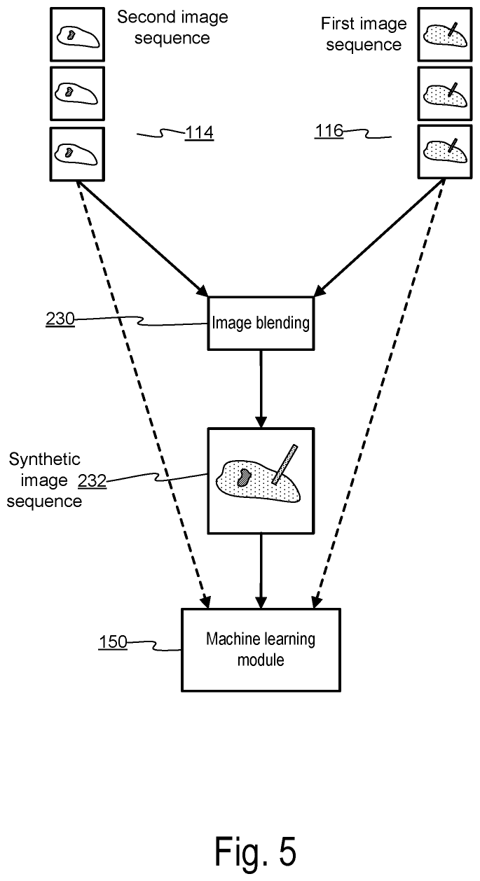

FIG. 5 shows an embodiment using image blending. In this embodiment, second images and time-corresponding first images are passed to an image blending module 230. Each second image received by the image blending module 230 is paired with one or more first images (for brevity, first images will be referred to in the singular) that are also received by the image blending module 230. The image blending module 230 may perform geometric transforms to geometrically align the first image and the second image (e.g., so that the images represent a same view of the scene (respectively corresponding pixels represent a same point of the scene). The image blending module 230 may create a synthetic image based on image data from the first image and the second image. For example, the image blending module 230 may perform feature detection, segmentation, etc. on both images, and may create a synthetic image 232 by combining features from both images. If two features in the respective images are determined to match, one might be selected for inclusion in the synthetic image 232 by matching the two features (e.g., based on position, shape, intensities, etc.) If a feature (e.g., an object or patch of tissue) is found in one image but not the other, the feature may be included in the synthetic image. In some implementations, one of the images may serve as an initial version of the synthetic image 232, and the initial version is modified according to content in the other image. In one implementation, both images are segmented, and the synthetic image 232 is formed by a union of the segments of both images.

The synthetic images 232 constructed from respective pairs of first and second images are passed to a machine learning module 150 which trains a model (if the machine learning module is a training algorithm) or produces predictions about the synthetic images 232. The predictions may be any of the types of predictions discussed above. In some embodiments, as indicated by the dashed arrows in FIG. 5, either or both of the first image sequences 114 and the second image sequences 116 are also passed to the machine learning module 150, thus providing additional training or prediction data. Moreover, feature detection and labeling may be performed on any of the image sequences supplied to the machine learning module 150.

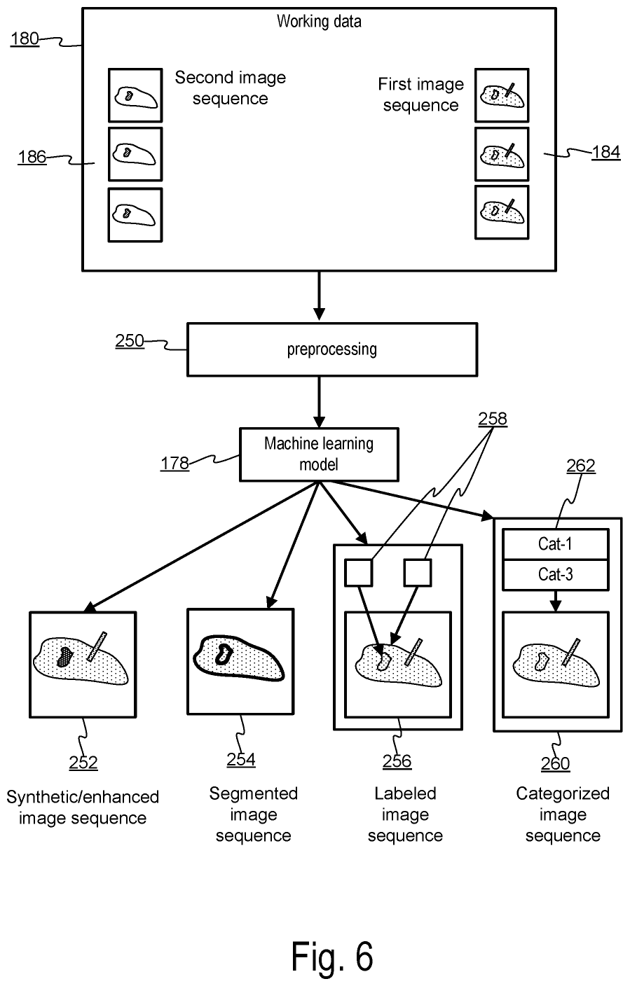

FIG. 6 shows examples of outputs of a trained machine learning model 178. A preprocessing module 250 may generate, from the working data 180, any of the variations of image sequences discussed above. For example, the preprocessing module 250 may output various combinations of labeled second images, synthetic images, labeled first images, segmented images, etc. The machine learning model 178 in turn outputs one or more predictions. For example, a prediction might be a synthetic image 252, which might include image data from a first image and a second image. For example, a synthetic image 252 might be a union of features from a first image and a second image. A synthetic image 252 might be an enhanced first (or second) image, for example with values of pixels changed to highlight features, form sharper or more uniform features, etc. Another possible output might be a segmented image 254, with segments identified by a separate bitmask, by enhancing pixels on the borders of segments, by coloring pixels within according to identified types of the segments, and so forth. Another possible output is a labeled image 256. A labeled image 256 may have predicted labels 258 of respective features (which themselves may be predictions), sets of tags of respective features, etc. Another possible output is a categorized image 260. The machine learning model 178 outputs one or more predicted category tags 262 (if any) for respective images. For example, the machine learning model might predict a stage of a medical procedure, a category of any feature detected in an image (e.g., a type of object present, a type of tissue or organ present, etc.), or others. The machine learning model 178 may predict geometry of the depicted scene, for example, the predicted depth of features or particular pixels, predicted distances between features, reconstructed three-dimensional geometry of the scene, etc. Any combinations of the above-mentioned predictions may be output.

As noted above, machine learning predictions informed by non-visible-spectrum image data may stand on their own as useful outputs. For example, predictions may be used for teaching, post-operative evaluation, identifying critical stages of a procedure, estimating anatomical dimensions, etc. During a medical procedure, notifications may be provided to operating-room personnel. For example, notifications may be rendered as sound or graphics, for example to inform personal of critical stages of a medical procedure, the presence or proximity of sensitive tissue or organs, recommended actions, etc.

As also noted above, machine learning predictions may also be used as inputs to other systems or software (including the computer-assisted medical system 188). For example, the predictions may be provided to an enhanced reality system (e.g., a virtual reality (VR) system or an augmented reality (AR) system), which may be implemented individually or by the computer-assisted medical system 188. A VR system may simulate a scene in three dimensions. Features such as objects may be enhanced. Sub-surface or intra-tissue features might be displayed, possibly conditionally, for example when a viewpoint is within a threshold distance of a feature. Features may be graphically labeled in a user interface. Predictions may be used for scene reconstruction, and so on. An AR system might display such graphics during a medical procedure for real-time visualization of the procedure. An AR or VR system might designate three-dimensional zones from which instruments or objects may be excluded. Predicted synthetic images might provide image data displayed by an AR or VR system (e.g., textures, colors, or intensities to be mapped to scene geometry or surfaces).

As mentioned, predictions may be provided to the computer-assisted medical system 188 to improve its functionality during a medical procedure. To illustrate, predictions may be used to control the manipulation of one or more instruments by the computer-assisted medical system 188. For example, predictions may inform instrument tracking, localization, or identification. Additionally or alternatively, predicted features may form the basis for exclusion-zones; the computer-assisted medical system 188 may automatically inhibit instruments from contacting certain types of tissue or anatomy or from moving into zones around predicted features. Additionally or alternatively, the computer-assisted medical system 188 may display predicted graphics as discussed above. For example, predicted segmentations or labels may be displayed. Various other operations may be based on predictions (e.g., presence of a particular type of anatomy) such as triggering video recording when specific anatomy is recognized based on the output of the machine learning model, user interface changes, rendering notifications, displaying information about predicted surgical stages, control of the light sources 110 or imaging device 112, and/or other peripheral events. In some embodiments, tissue/structure models may be built from partial views (e.g., obscured views of a biliary tree during dissection) to help a surgeon better track anatomy.

As has been described, the imaging device 112 and/or image processing system 126 may be associated in certain examples with a computer-assisted medical system used to perform a medical procedure on a body (whether alive or not). To illustrate, FIG. 7 shows an example of a computer-assisted medical system 700 that may be used to perform various types of medical procedures including surgical and/or non-medical procedures. The imaging device 112 and the image processing system 126 may be part of, or supplement, the computer-assisted medical system 188.

As shown, the computer-assisted medical system 188 may include a manipulator assembly 702 (a manipulator cart is shown in FIG. 7), a user control apparatus 704, and an auxiliary apparatus 706, all of which are communicatively coupled to each other. The computer-assisted medical system 188 may be utilized by a medical team to perform a computer-assisted medical procedure or other similar operation on a body of a patient 708 or on any other body as may serve a particular implementation. As shown, the medical team may include a first user 710-1 (such as a surgeon for a medical procedure), a second user 710-2 (such as a patient-side assistant), a third user 710-3 (such as another assistant, a nurse, a trainee, etc.), and a fourth user 710-4 (such as an anesthesiologist for a medical procedure), all of whom may be collectively referred to as users 710, and each of whom may control, interact with, or otherwise be a user of the computer-assisted medical system 188. More, fewer, or alternative users may be present during a medical procedure as may serve a particular implementation. For example, team composition for different medical procedures, or for non-medical procedures, may differ and include users with different roles.

While FIG. 7 illustrates an ongoing minimally invasive medical procedure such as a minimally invasive medical procedure, it will be understood that the computer-assisted medical system 188 may similarly be used to perform open medical procedures or other types of operations. For example, operations such as exploratory imaging operations, mock medical procedures used for training purposes, and/or other operations may also be performed.

As shown in FIG. 7, the manipulator assembly 702 may include one or more manipulator arms 712 (e.g., manipulator arms 712-1 through 712-4) to which one or more instruments may be coupled. The instruments may be used for a computer-assisted medical procedure on patient the 708 (e.g., in a surgical example, by being at least partially inserted into the patient 708 and manipulated within the patient 708). While the manipulator assembly 702 is depicted and described herein as including four manipulator arms 712, the manipulator assembly 702 may include a single manipulator arm 712 or any other number of manipulator arms. While the example of FIG. 7 illustrates the manipulator arms 712 as being robotic manipulator arms, it will be understood that, in some examples, one or more instruments may be partially or entirely manually controlled, such as by being handheld and controlled manually by a person. For instance, these partially or entirely manually controlled instruments may be used in conjunction with, or as an alternative to, computer-assisted instrumentation that is coupled to the manipulator arms 712 shown in FIG. 7.

During the medical operation, the user control apparatus 704 may be configured to facilitate teleoperational control by the user 710-1 of the manipulator arms 712 and instruments attached to the manipulator arms 712. To this end, the user control apparatus 704 may provide the user 710-1 with imagery of an operational area associated with patient 708 as captured by an imaging device. To facilitate control of instruments, user control apparatus 704 may include a set of master controls. These master controls may be manipulated by the user 710-1 to control movement of the manipulator arms 712 or any instruments coupled to the manipulator arms 712.

The auxiliary apparatus 706 may include one or more computing devices configured to perform auxiliary functions in support of the medical procedure, such as providing insufflation, electrocautery energy, illumination or other energy for imaging devices, image processing, or coordinating components of computer-assisted medical system 188. In some examples, the auxiliary apparatus 706 may be configured with a display monitor 714 configured to display one or more user interfaces, or graphical or textual information in support of the medical procedure. In some instances, the display monitor 714 may be implemented by a touchscreen display and provide user input functionality. Augmented content provided by a region-based augmentation system may be similar, or differ from, content associated with the display monitor 714 or one or more display devices in the operation area (not shown).

The manipulator assembly 702, user control apparatus 704, and auxiliary apparatus 706 may be communicatively coupled one to another in any suitable manner. For example, as shown in FIG. 7, the manipulator assembly 702, user control apparatus 704, and auxiliary apparatus 706 may be communicatively coupled by control lines 716, which may represent any wired or wireless communication link as may serve a particular implementation. To this end, the manipulator assembly 702, user control apparatus 704, and auxiliary apparatus 706 may each include one or more wired or wireless communication interfaces, such as one or more local area network interfaces, Wi-Fi network interfaces, cellular interfaces, and so forth.

In certain embodiments, one or more of the processes described herein may be implemented at least in part as instructions embodied in a non-transitory computer-readable medium and executable by one or more computing devices. In general, a processor (e.g., a microprocessor) receives instructions, from a non-transitory computer-readable medium, (e.g., a memory, etc.), and executes those instructions, thereby performing one or more processes, including one or more of the processes described herein. Such instructions may be stored and/or transmitted using any of a variety of known computer-readable media.

A computer-readable medium (also referred to as a processor-readable medium) includes any non-transitory medium that participates in providing data (e.g., instructions) that may be read by a computer (e.g., by a processor of a computer). Such a medium may take many forms, including, but not limited to, non-volatile media, and/or volatile media. Non-volatile media may include, for example, optical or magnetic disks and other persistent memory. Volatile media may include, for example, dynamic random-access memory (“DRAM”), which typically constitutes a main memory. Common forms of computer-readable media include, for example, a disk, hard disk, magnetic tape, any other magnetic medium, a compact disc read-only memory (“CD-ROM”), a digital video disc (“DVD”), any other optical medium, random access memory (“RAM”), programmable read-only memory (“PROM”), electrically erasable programmable read-only memory (“EPROM”), FLASH-EEPROM, any other memory chip or cartridge, or any other tangible medium from which a computer can read.

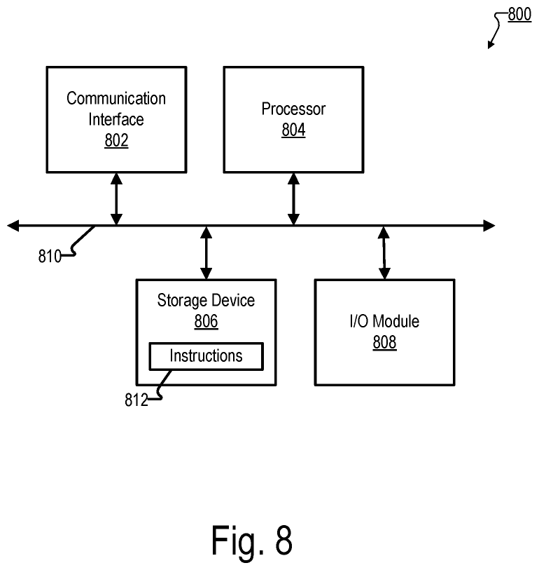

FIG. 8 shows an illustrative computing device 800 that may be specifically configured to perform one or more of the processes described herein. Any of the systems, computing devices, and/or other components described herein may be implemented by the computing device 800.

As shown in FIG. 8, the computing device 800 may include a communication interface 802, a processor 804, a storage device 806, and an input/output (“I/O”) module 808 communicatively connected one to another via a communication infrastructure 810. While an illustrative computing device 800 is shown in FIG. 8, the components illustrated in FIG. 8 are not intended to be limiting. Additional or alternative components may be used in other embodiments. The computing device 800 may be a virtual machine or may include virtualized components. Components of the computing device 800 shown in FIG. 8 will now be described in additional detail.

The communication interface 802 may be configured to communicate with one or more computing devices. Examples of the communication interface 802 include, without limitation, a wired network interface (such as a network interface card), a wireless network interface (such as a wireless network interface card), a modem, an audio/video connection, and any other suitable interface.

The processor 804 generally represents any type or form of processing unit capable of processing data and/or interpreting, executing, and/or directing execution of one or more of the instructions, processes, and/or operations described herein. The processor 804 may perform operations by executing computer-executable instructions 812 (e.g., an application, software, code, and/or other executable data instance) stored in the storage device 806.

The storage device 806 may include one or more data storage media, devices, or configurations and may employ any type, form, and combination of data storage media and/or device. For example, the storage device 806 may include, but is not limited to, any combination of the non-volatile media and/or volatile media described herein. Electronic data, including data described herein, may be temporarily and/or permanently stored in the storage device 806. For example, data representative of computer-executable instructions 812 configured to direct the processor 804 to perform any of the operations described herein may be stored within the storage device 806. In some examples, data may be arranged in one or more databases residing within the storage device 806.

The I/O module 808 may include one or more I/O modules configured to receive user input and provide user output. The I/O module 808 may include any hardware, firmware, software, or combination thereof supportive of input and output capabilities. For example, the I/O module 808 may include hardware and/or software for capturing user input, including, but not limited to, a keyboard or keypad, a touchscreen component (e.g., touchscreen display), a receiver (e.g., an RF or infrared receiver), motion sensors, and/or one or more input buttons.

The I/O module 808 may include one or more devices for presenting output to a user, including, but not limited to, a graphics engine, a display (e.g., a display screen), one or more output drivers (e.g., display drivers), one or more audio speakers, and one or more audio drivers. In certain embodiments, the I/O module 808 is configured to provide graphical data to a display for presentation to a user. The graphical data may be representative of one or more graphical user interfaces and/or any other graphical content as may serve a particular implementation.

In the preceding description, various exemplary embodiments have been described with reference to the accompanying drawings. It will, however, be evident that various modifications and changes may be made thereto, and additional embodiments may be implemented, without departing from the scope of the invention as set forth in the claims that follow. For example, certain features of one embodiment described herein may be combined with or substituted for features of another embodiment described herein. The description and drawings are accordingly to be regarded in an illustrative rather than a restrictive sense.

1. A system comprising:
a memory storing instructions; and
one or more processors communicatively coupled to the memory and configured to execute the instructions to perform a process comprising:
accessing a first image sequence captured by an imaging device during a medical procedure, the first image sequence comprising first images, the first images based on illumination of a scene associated with the medical procedure using visible-spectrum light;
accessing a second image sequence captured by the imaging device during the medical procedure, the second image sequence comprising second images, the second images comprising non-visible-spectrum images based on illumination of the scene using non-visible spectrum light;
detecting one or more features in the second images;
applying one or more labels to the second images to generate labeled second images, the one or more labels indicating the one or more features; and
processing the first images and the labeled second images using a machine learning module.

2. The system according to claim 1, wherein the second images are based on sensing of infrared light.

3. The system according to claim 2, wherein the infrared light comprises light emitted by illuminated fluorophores.

4. The system according to claim 1, wherein the machine learning module comprises a machine learning algorithm, and wherein the processing comprises training, by the machine learning algorithm, a machine learning model based on the first image sequence and the second image sequence.

5. The system according to claim 1, wherein the machine learning module comprises a trained machine learning model, and wherein the processing comprises generating, by the trained machine learning model, a prediction based on the first image sequence and the second image sequence.

6. The system according to claim 5, wherein the prediction comprises one or more of: a predicted image, a predicted label indicative of features in one or more of the first image sequence or the second image sequence, an image segmentation, a predicted stage of a medical procedure, or a predicted geometry corresponding to the scene.

7. The system according to claim 5, the process further comprising providing the prediction to a computer-assisted medical system that performs an operation based on the prediction.

8. The system according to claim 1, wherein the processing comprises generating labels for the first image sequence based on the second image sequence.

9. The system according to claim 8, wherein the processing further comprises training a machine learning model based on the first image sequence and the labels.

10. A system comprising:
a memory storing instructions; and
one or more processors communicatively coupled to the memory and configured to execute the instructions to perform a process comprising:
accessing a first image sequence captured by an imaging device during a medical procedure, the first image sequence comprising first images, the first images based on illumination of a scene associated with the medical procedure using visible-spectrum light;
providing the first images to a trained machine learning model, wherein the trained machine learning model has been trained using a second image sequence comprising second images based on illumination of the scene using non-visible-spectrum light; and
performing, based on an output of the trained machine learning model, an operation with respect to the first image sequence.

11. The system according to claim 10, the process further comprising generating, based on the output of the trained machine learning model, a prediction for use with a computer-assisted medical system.

12. The system according to claim 11, the process further comprising displaying, based on the prediction, a user interface by way of a display of the computer-assisted medical system.

13. The system according to claim 11, the process further comprising controlling, based on the prediction, a movement of a component of the computer-assisted medical system.

14. The system according to claim 10, wherein the output comprises a modified version of an image in the first images, and wherein the operation comprises displaying the modified version of the image.

15. The system according to claim 14, wherein the modified version of the image comprises a segmentation of the image.

16. The system according to claim 10, wherein the operation comprises one or more of: segmenting an image in the first images, labeling the image, categorizing the image, reconstructing a geometry or measure of the scene, or identifying a feature depicted in the image.

17. The system according to claim 16, wherein the output comprises a label associated with the image, and wherein the label comprises an indication of at least one of a type of tissue, an identification of an organ, or an indication of a type of object.

18-24. (canceled)

25. A system comprising:
a memory storing instructions; and
one or more processors communicatively coupled to the memory and configured to execute the instructions to perform a process comprising:
accessing a first image sequence captured by an imaging device during a medical procedure, the first image sequence comprising first images, the first images based on illumination of a scene associated with the medical procedure using visible-spectrum light;
providing the first images to a trained machine learning model, wherein the trained machine learning model has been trained using a second image sequence comprising second images based on illumination of the scene using non-visible-spectrum light; and
generating, based on an output of the trained machine learning model, a prediction.

26-27. (canceled)

28. The system according to claim 25, wherein the process further comprises performing, based on the prediction, an operation with respect to a computer-assisted medical system.

29. The system according to claim 28, wherein the performing the operation comprises one or more of displaying a graphical user interface by way of a display of the computer-assisted medical system, displaying an image included in the first images by way of the display of the computer-assisted medical system, or controlling a movement of a component of the computer-assisted medical system.

30-43. (canceled)
